(19) United States
(12) Patent Application Publication
(10) Pub. No.: US 2007/0203235 A1
(43) Pub. Date: Aug. 30, 2007

(54) Method for preventing or treating anemia

(76) Inventors: Francisco J. Rosales, Newburgh, IN (US); Joshua C. Anthony, Evansville, IN (US); James T. Brenna, Ithaca, NY (US); Andrea Tseng Hsieh, Wheaton, IL (US)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(21) Appl. No.: 11/364,619

(22) Filed: Feb. 28, 2006

(51) Int. Cl. A61K 31/202

(52) U.S. Cl. 514/560

(57) ABSTRACT

The present invention is directed to a novel method for preventing or treating anemia in a subject. The method comprises administration of a therapeutically effective amount of DHA and ARA to the subject.

### BACKGROUND OF THE INVENTION

(1) Field of the Invention

The present invention relates generally to a method for preventing or treating anemia.

(2) Description of the Related Art

Human blood contains three types of cells—red blood cells, white blood cells, and platelets—all of which circulate throughout the body. Red blood cells (RBC) contain hemoglobin (Hb), a red, iron-rich protein that carries oxygen from the lungs to all of the body's muscles and organs where it reacts to provide the energy the body needs for its normal activities. When the number of red blood cells or the amount of hemoglobin they contain fall below normal, the body receives less oxygen and generates less energy than it needs to function properly. This condition in general is referred to as anemia.

Almost 100 different types of anemia are recognized, each having different causes. Among the causes of anemia are inadequate production of red blood cells, a destruction of red blood cells (hemolytic anemia), or a substantial blood loss. Anemia is often linked with an iron deficiency, but other causes of anemia can include a vitamin B12 deficiency, a foliate deficiency, inherited disorders, renal disease, or liver disease.

Symptoms of anemia include shortness of breath, palpitations of the heart, heart murmurs, lethargy, and increased fatigue. If left untreated, anemia may cause more serious problems. When the number of red blood cells decreases, the heart works harder by pumping more blood to deliver more oxygen throughout the body. If the heart works too hard, it can develop a rapid heartbeat (tachycardia), and/or another serious condition known as left ventricular hypertrophy (LVH), an enlargement of the heart muscle that can lead to heart failure.

In addition to adults, as many as 20% of children in the United States and 80% of children in developing countries will become anemic at some point by the age of 18 years. Martin, P. L., et al. The Anemias, Principles and Practices of Pediatrics, 1657 (2d ed., Lippincott 1994). Neonatal anemia is a physiological condition characterized by a postnatal reduction in red blood cell mass or Hb concentration. Clinical signs and symptoms include poor feeding, dyspnea, tachycardia, tachypnea, diminished activity, and pallor as infants struggle to compensate for inadequate oxygenation.

The “physiologic anemia of infancy” is a specific postnatal concern during early infancy where neonates tolerate remarkably low levels of Hb without exhibiting other abnormalities. This fall in Hb is not fully understood, but is believed to result from a decrease in hematopoietic activity, red blood cell mass, and shortened red blood cell survival as infants adapt to a variety of complex changes in oxygen transport and delivery triggered at birth. Infants born with widely varying hemoglobin values reach similarly low values before the natural onset of active erythropoiesis.

Though not the only cause, a common cause for anemia among infants and children is an iron deficiency. At birth, most term infants have 75 mg of elemental iron per kilogram of body weight, found primarily as Hb (75%), but also as storage (15%) and tissue protein iron (10%). Am. Acad. on Pediatr., Comm. on Nutrition, Iron Fortification of Infant Formulas, Pediatr. 104:119-123 (1999). Typically, during the first 4 postnatal months, excess fetal red blood cells break down and the infant is able to retain the iron. This iron is used, along with dietary iron, to support the expansion of the red blood cell mass as the infant grows. The estimated iron requirement for the term infant to meet this demand and also maintain adequate iron storage is about 1 mg/kg per day.

Because a newborn term infant accretes more than 80% of its iron during the third trimester of gestation, preterm infants must accrete more iron postnatally to “catch up” to their term counterparts during the first year. Thus, the iron intake requirements for preterm infants range from 2 mg/kg per day for infants with birth weights between 1500 and 2500 g to4 mg/kg per day for infants weighing less than 1500 g at birth.

Due to these high iron requirements, it is very important that postnatal dietary iron sources be well-absorbed by the infant. Although iron concentrations in human milk are relatively low (approximately 0.3 mg/L), the iron contained in human milk has been shown to be absorbed better by infants than the iron in either cow's milk or soy milk. For example, between 50% and 70% of iron from human milk is absorbed into the infant body, compared with typically less than 12% of iron from cow's milk-based formula. The percentage of iron absorbed from soy-based formula is even lower than that from cow's milk formula and ranges from less than 1% to 7%. The high bioavailabilty of iron in human milk is a factor in experts' recommendations that infants be breast-fed until at least one year of age.

Despite the benefits of breastfeeding, not all mothers are willing or able to breastfeed. Currently, most infants in the United States are not breastfed beyond three months of age. Because the iron sources in infant formulas are not as well absorbed as the iron sources in breast milk, infant formulas must contain higher quantities of iron than breast milk in order to deliver an equal amount of bioavailable iron to the infant. This has led to the development of iron-fortified infant formulas. In the United States, iron concentrations in iron-fortified formulas range from 10 mg/L to 12 mg/L. In Europe, infant formula tends to contain 4 mg/L to 7 mg/L of iron.

Unfortunately, iron-fortified infant formulas are often avoided by consumers due to worries that excess iron will cause gastrointestinal distress for their infant. Consumers also continue to have concerns about high levels of iron interfering with the immune system. Therefore, many consumers still prefer to use a low-iron infant formula, placing their infants at risk for anemia.

Because anemia is commonly associated with an iron deficiency, iron supplements are often prescribed to remedy the condition. The body can release only a certain amount of excess iron per day, however. If individuals consume excessive amounts of iron that the body is unable to release, the body may store the excess iron in cells of the liver, heart, pancreas, and other organs. This condition is known as hemochromatosis. If left untreated, hemochromatosis can lead to diabetes, joint pain, abnormal heart rhythms, heart failure, cirrhosis of the liver, or liver failure.

Therefore, it would be beneficial to provide a method of treatment or prevention for anemia that does not involve the consumption of iron supplements. Because there are multiple types of anemia that are unrelated to iron absorption, and for which an iron supplement would be ineffective and potentially dangerous, it would be beneficial to provide a composition that can prevent or treat multiple forms of anemia without supplementing the diet with iron. In addition, it would be beneficial to provide an infant formula or children's nutritional product containing such a composition in order to prevent or treat multiple forms of anemia in infants and children.

### SUMMARY OF THE INVENTION

Briefly, the present invention is directed to a novel method for preventing or treating anemia in a subject, the method comprising administering to the subject a therapeutically effective amount of DHA and ARA. The invention is also directed to a novel method for increasing the red blood cell count in a subject, the method comprising administering to the subject a therapeutically effective amount of DHA and ARA. The invention is further directed to a novel method for increasing the hemoglobin concentration in a subject, the method comprising administering to the subject a therapeutically effective amount of DHA and ARA.

The present invention is additionally directed to a method for elevating hematocrit values in a subject, the method comprising administering to the subject a therapeutically effective amount of DHA and ARA. Further, the present invention is directed to a novel method for promoting accelerated erythropoiesis in an infant, the method comprising administering to the infant a therapeutically effective amount of DHA and ARA. Additionally, the present invention is directed to a novel method for enhancing the ability of a subject to absorb iron, the method comprising administering to the infant a therapeutically effective amount of DHA and ARA.

Among the several advantages found to be achieved by the present invention, is that it provides a method for preventing or treating multiple forms of anemia without the necessity of administering excessive amounts of iron.

### BRIEF DESCRIPTION OF THE DRAWINGS

For a more complete understanding of the present invention, reference is now made to the following descriptions taken in conjunction with the accompanying drawings.

FIG. 1 is a graph illustrating the effects of DHA and ARA supplementation on RBC counts.

FIG. 2 is a graph illustrating the effects of DHA and ARA supplementation on Hb counts.

FIG. 3 is a graph illustrating the effect of DHA and ARA supplementation on hematocrit values.

FIG. 4 is a graph illustrating the effect of DHA and ARA supplementation on RBC distribution width.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

As used herein, the term “treating” means ameliorating, improving or remedying a disease, disorder, or symptom of a disease or condition.

The term “preventing” means to stop or hinder a disease, disorder, or symptom of a disease or condition through some action.

The terms “therapeutically effective amount” refer to an amount that results in an improvement or remediation of the disease, disorder, or symptoms of the disease or condition.

The term “subject” for the purposes of the present invention includes any human or animal subject. The subject is preferably one that is in need of the prevention of or treatment of anemia. The subject is typically a mammal. “Mammal”, as that term is used herein, refers to any animal classified as a mammal, including humans, domestic and farm animals, and zoo, sports, or pet animals, such as dogs, horses, cats, cattle, etc.,

The term “infant” means a postnatal human that is less than about 1 year of age.

As used herein, the term “anemia” can be defined as any condition in which the number of red blood cells or the amount of hemoglobin contained within those cells is less than accepted age-specific and gender-specific limits.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, the inventors have discovered a novel method for preventing or treating anemia in a subject, which comprises administering a therapeutically effective amount of docosahexaenoic acid (DHA) and arachidonic acid (ARA) to the subject. In fact, it has been shown in the present invention that the administration of DHA and ARA increases red blood cell, hemoglobin, and hematocrit values by between 12% and 15%, thus preventing and/or alleviating anemia.

DHA and ARA are long chain polyunsaturated fatty acids (LCPUFA) which have been shown to contribute to the health and growth of infants. Specifically, DHA and ARA have been shown to support the development and maintenance of the brain, eyes and nerves of infants. Birch, E., et al., A Randomized Controlled Trial of Long-Chain Polyunsaturated Fatty Acid Supplementation of Formula in Term Infants after Weaning at 6 Weeks of Age, Am. J. Clin. Nutr. 75:570-580 (2002). Clandinin, M., et al., Formulas with Docosahexaenoic Acid (DHA) and Arachidonic Acid (ARA) Promote Better Growth and Development Scores in Very-Low-Birth-Weight Infants (VLBW), Pediatr. Res.51:187A-188A (2002). DHA and ARA are typically obtained through breast milk in infants that are breast-fed. In infants that are formula-fed, however, DHA and ARA must be supplemented into the diet.

While it has been shown that DHA and ARA are beneficial to the development of brain, eyes and nerves in infants, DHA and ARA have not previously been shown to have any effect on anemia. The positive effects of DHA and ARA on anemia that were discovered in the present invention were surprising and unexpected.

In some embodiments of the present invention, the subject is in need of prevention and/or treatment of anemia. The subject can be a human subject who is at risk for developing anemia. The subject can be at risk due to genetic predisposition, lifestyle, diet, inherited disorders, vitamin or mineral deficiencies, diseases or disorders, and the like. For example, a subject having certain renal or liver diseases is one at risk for developing anemia.

In certain embodiments of the present invention, the subject in need of prevention and/or treatment for anemia is an infant. In a particular embodiment, the subject in need of prevention and/or treatment for anemia is a preterm infant. As another example, a preterm infant may be at risk for developing anemia because more than 80% of iron accretion occurs during the third trimester of gestation, a period of development cut short for preterm infants.

In the present invention, the form of administration of DHA and ARA is not critical, as long as a therapeutically effective amount is administered to the subject. In some embodiments, the DHA and ARA are administered to a subject via tablets, pills, encapsulations, caplets, gelcaps, capsules, oil drops, or sachets. In another embodiment, the DHA and ARA are added to a food or drink product and consumed.

In some embodiments of the invention, the DHA and ARA are supplemented into the diet of an infant or child for the purpose of preventing or treating anemia. In this embodiment, DHA and ARA can be supplemented into an infant formula or a children's nutritional product which can then be fed to an infant or child.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, canola oil, corn oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil® with Iron, Lactofree®, Nutramigen®, Pregestimil®, and ProSobee® (available from Mead Johnson & Company, Evansville, Ind., U.S.A.) may be supplemented with suitable levels of DHA and ARA and used in practice of the method of the invention. Additionally, Enfamil® LIPIL®, which contains effective levels of DHA and ARA, is commercially available and may be utilized in the present invention.

The method of the invention requires the administration of a combination of DHA and ARA. In this embodiment, the weight ratio of ARA:DHA can be from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5.

In certain embodiments of the invention, the level of DHA is between about 0.32% and 0.96% of fatty acids. In other embodiments of the invention, the level of ARA is between 0.0% and 0.64% of fatty acids. Thus, in certain embodiments of the invention, DHA alone can treat or prevent anemia in a subject.

The effective amount of DHA in an embodiment of the present invention is typically from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

When used, the effective amount of ARA in an embodiment of the present invention is typically from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The amount of DHA in infant formulas for use in the present invention typically varies from about 2 mg/100 kilocalories (kcal) to about 100 mg/100 kcal. In another embodiment, the amount of DHA varies from about 5 mg/100 kcal to about 75 mg/100 kcal. In yet another embodiment, the amount of DHA varies from about 15 mg/100 kcal to about 60 mg/100 kcal.

When used, the amount of ARA in infant formulas for use in the present invention typically varies from about 4 mg/100 kilocalories (kcal) to about 100 mg/100 kcal. In another embodiment, the amount of ARA varies from about 10 mg/100 kcal to about 67 mg/100 kcal. In yet another embodiment, the amount of ARA varies from about 20 mg/100 kcal to about 50 mg/100 kcal. In a particular embodiment, the amount of ARA varies from about 30 mg/100 kcal to about 40 mg/100 kcal.

The infant formula supplemented with oils containing DHA and ARA for use in the present invention can be made using standard techniques known in the art. For example, an equivalent amount of an oil which is normally present in infant formula, such as high oleic sunflower oil, may be replaced with DHA and ARA.

The source of the ARA and DHA can be any source known in the art such as fish oil, single cell oil, egg yolk lipid, brain lipid, and the like. The DHA and ARA can be in natural form, provided that the remainder of the LCPUFA source does not result in any substantial deleterious effect on the infant. Alternatively, the DHA and ARA can be used in refined form.

The LCPUFA used in the present invention may or may not contain EPA. In certain embodiments, the LCPUFA used in the invention contains little or no eicosapentaenoic acid (EPA). For example, in certain embodiments, the infant formulas used herein contain less than about 20 mg/100 kcal EPA. In other embodiments the infant formulas used herein contain less than about 10 mg/100 kcal EPA. In still other embodiments the infant formulas used herein contain less than about 5 mg/100 kcal EPA. In a particular embodiment, the infant formulas used herein contain substantially no EPA.

Sources of DHA and ARA may be single cell oils as taught in U.S. Pat. Nos. 5,374,657, 5,550,156, and 5,397,591, the disclosures of which are incorporated herein by reference in their entirety.

In an embodiment of the present invention, the DHA and ARA are supplemented into the diet of an infant from birth until the infant reaches about one year of age. In another embodiment of the invention, the DHA and ARA are supplemented into the diet of an infant from birth until the infant reaches about two years of age. In other embodiments, the DHA and ARA are supplemented into the diet of a subject for the lifetime of the subject. The present invention can be used to treat clinically healthy subjects as well as subjects having some form of anemia.

In the present invention, DHA and ARA supplementation is effective in treating or preventing many types of anemia, including, but not limited to: hemolytic anemia, microangiopathic hemolytic anemia, hypersplenism, pyruvate kinase deficiency, dyserythropoietic anemia, spherocytosis, sideroblastic anemia, autoimmune hemolytic anemia, sickle cell anemia, thalassemia, Glucose-6-phosphate dehydrogenase (G6PD)-deficient anemia, liver disease, renal disease, pernicious anemia, aplastic anemia, or various vitamin or nutrient-deficiencies, such as vitamin B12, B2, B6, C, A, D, E, or K, iron, folic acid, zinc, copper, calcium, or protein.

As will be seen in the examples, benefits of the present invention include the promotion of RBC synthesis, enhancement of the life span of the fetal erythrocytes, increasing the incorporation of dietary iron into RBC, and concomitantly, reducing the iron needs in a subject.

In certain embodiments, the invention provides a method for increasing the red blood cell count in a subject, the method comprising administering to the subject a therapeutically effective amount of DHA and ARA. In other embodiments, the invention provides a method for increasing the hemoglobin concentration in a subject, the method comprising administering to the subject a therapeutically effective amount of DHA and ARA. In further embodiments, the invention provides a method for elevating hematocrit values in a subject, the method comprising administering to the subject a therapeutically effective amount of DHA and ARA. In a particular embodiment, the invention provides a method for promoting accelerated erythropoiesis in an infant, the method comprising administering to the infant a therapeutically effective amount of DHA and ARA. Additionally, the present invention provides a method for enhancing the ability of a subject to absorb iron, the method comprising administering to the infant a therapeutically effective amount of DHA and ARA.

In any of these embodiments, the subject is any human or animal subject. In some embodiments, the subject is in need of prevention and/or treatment of anemia. The subject can be a human subject who is at risk for developing anemia. The subject can be at risk due to genetic predisposition, lifestyle, diet, inherited disorders, vitamin or mineral deficiencies, diseases or disorders, and the like. In certain embodiments of the present invention, the subject in need of prevention and/or treatment for anemia is an infant. In a particular embodiment, the subject in need of prevention and/or treatment for anemia is a preterm infant.

The present invention is also directed to the use of DHA and ARA for the preparation of a medicament for the prevention or treatment of anemia. In this embodiment, the DHA and ARA can be used to prepare a medicament for the prevention or treatment of anemia in any human or animal subject. For example, the medicament could be used to prevent or treat anemia in domestic, farm, zoo, sports, or pet animals, such as dogs, horses, cats, cattle, and the like. In some embodiments, the subject is in need of prevention and/or treatment of anemia.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### General Procedures for Examples

Materials and General Procedures Used to Carry Out the Present Invention and to Measure the Results are Described Below:

Animal Testing:

Animal work took place at the Southwest Foundation for Biomedical Research (SFBR) located in San Antonio, Tex. and protocols were approved by the SFBR Institutional Animal Care and Use Committee. Fourteen pregnant baboons delivered spontaneously around 182 days gestation. Baboon neonate characteristics are summarized in Table 1.

TABLE 1
Baboon Neonate Characteristics

| | |
|---|---|
| Number of animals (n) | 14 |
| Gender | 10F, 4M |
| Conceptional age at delivery (d) | 182 ± 6 |
| Birth weight (g) | 860 ± 151 |
| Weight at 12 weeks (g) | 1519 ± 281 |
| Weight gain (g) | 658 ± 190.4 |

Neonates were transferred to the nursery within 24 hours of birth and randomized to one of three diet groups. Animals were assigned to one of the following formulas: Control (C), unsupplemented; supplemented with 0.32% DHA and 0.64% ARA (L) and supplemented with 0.96% DHA and 0.64% ARA (L3). C and L are commercially available human infant formulas (Enfamil® and Enfamil® Lipilυ, respectively), and all diets provided 1.8 mg/100 cal of iron. Formulas were provided by Mead-Johnson Nutritionals (Evansville, Ind.). Animals were housed in enclosed incubators until 2 weeks of age and-then moved to individual stainless steel cages in a controlled access nursery. Room temperature was maintained between 76° C. and 82° C., with a 12 hour light/dark cycle.

Neonatal growth was assessed using body weight measurements, recorded two or three times weekly. Head circumference and crown-rump length data were obtained weekly for each animal.

Blood was obtained via unsedated femoral venipuncture in fasted animals between 07:00 and 08:30. Hematological measurements were made on whole blood collected in potassium ethylenediaminetetraacetic acid (EDTA) microtainer tubes at 2, 4, 8, 10, and 12 weeks of age.

Measurement and Analysis of Data:

Parameters evaluated included white blood cell (WBC) counts, RBC counts, Hb concentrations, hematocrit, mean cell volume (MCV), mean cell hemoglobin (MCH), mean cell hemoglobin concentrations (MCHC), and red blood cell distribution width (RDW). Red cell indices MCV, MCH, MCHC and RDW are calculations based on the relationship between RBC, hemoglobin and hematocrit. Measurements were determined using a Coulter MAXM autoloader instrument (Beckman Coulter, Inc., Fullerton, Calif.).

Data are expressed as mean ±SD. Hematological values were evaluated using a random coefficient regression model to detect effects of LCPUFA supplementation. For every blood parameter, a slope and intercept was determined for each subject. Diet treatment was the fixed effect and random effects included subject, age, and the age/diet interaction. Regression analysis calculated intercepts using postnatal age—2 weeks, the initial sampling time point. Using an analysis of covariance, slopes were compared between diet groups with the baseline C group as the covariate. Anthropometric measurements were also assessed using a regression model to examine systematic effects of diet over time. Statistical analyses were performed using SAS for Windows 9.1 (SAS Institute, Cary, N.C.), with significance declared at p<0.05.

### EXAMPLE 1

This example describes the results of DHA and ARA supplementation in treating or preventing anemia in neonatal baboons.

Growth outcomes were assessed using animal body weight, head circumference and crown-rump length. Statistical analyses revealed no significant differences among diet treatments (p>0.37). Anthropometric measurements indicated normal neonatal growth and physical development.

Selected hematologic data from 2 to 12 weeks of age (mean ±SD) are shown in Tables 2-5.

TABLE 2
Clinical hematology reference values at 2 weeks of age for
LCPUFA supplemented term baboon neonates (range, mean ± SD).

| Diet | C | L | L3 |
|---|---|---|---|
| WBC (×10³) 4.6-9.6 | 6.73 ± 0.91 | 6.67 ± 0.31 | 7.30 ± 2.52 |
| RBC (×10⁵) 4.4-6.04 | 5.03 ± 0.47 | 5.76 ± 0.36 | 5.84 ± 0.03 |
| Hemoglobin (g/dl) | 14.10 ± 0.94 | 16.00 ± 0.66 | 16.33 ± 0.47 |
| 12.7-16.7 | | | |
| Hematocrit (%) 37.2- | 42.58 ± 3.69 | 49.87 ± 2.44 | 50.53 ± 0.23 |
| 52.0 | | | |
| MCV (fl) 80.1-89.4 | 84.80 ± 3.80 | 86.53 ± 1.29 | 86.53 ± 0.85 |
| MCH (pg) 26.2-28.8 | 28.05 ± 1.25 | 27.77 ± 0.55 | 28.00 ± 0.78 |
| MCHC (g/dl) 31.4- | 33.13 ± 0.79 | 32.07 ± 0.21 | 32.37 ± 0.87 |
| 34.1 | | | |
| ROW (%) 11.7-14.0 | 12.33 ± 0.61 | 13.17 ± 0.21 | 13.50 ± 0.44 |

TABLE 3
Clinical hematology reference values at 4 weeks of age for
LCPUFA supplemented term baboon neonates (range, mean ± SD.

| Diet | C | L | L3 |
|---|---|---|---|
| WBC (×10³) 6.1-13.4 | 9.83 ± 2.68 | 8.70 ± 2.15 | 8.53 ± 0.84 |
| RBC (×10⁶) 4.64-5.8 | 4.94 ± 0.09 | 5.24 ± 0.40 | 5.38 ± 0.38 |
| Hemoglobin (g/dl) | 13.08 ± 0.66 | 13.73 ± 0.69 | 14.38 ± 0.74 |
| 12.1-15.2 | | | |
| Hematocrit (%) 36.9- | 40.03 ± 2.10 | 42.70 ± 2.83 | 45.05 ± 2.75 |
| 45.9 | | | |
| MCV (fl) 76.4-86.1 | 81.08 ± 3.27 | 81.53 ± 1.15 | 83.85 ± 1.73 |
| MCH (pg) 25.1-27.7 | 26.53 ± 0.96 | 26.23 ± 0.80 | 26.83 ± 1.03 |
| MCHC (g/dl) 31.3- | 32.70 ± 0.22 | 32.15 ± 0.64 | 31.95 ± 0.70 |
| 33.1 | | | |
| RDW (%) 10.8-13.3 | 11.45 ± 0.47 | 12.43 ± 0.26 | 13.05 ± 0.31 |

TABLE 4
Clinical hematology reference values at 8 weeks of age for
LCPUFA supplemented term baboon neonates (range, mean ± SD.

| Diet | C | L | L3 |
|---|---|---|---|
| WBC (×10³) 4.4-11.4 | 8.98 ± 2.84 | 7.98 ± 1.68 | 9.16 ± 1.35 |
| RBC (×10⁶) 4.76-5.89 | 4.97 ± 0.13 | 5.10 ± 0.39 | 5.54 ± 0.27 |
| Hemoglobin (g/dl) | 12.28 ± 0.29 | 12.63 ± 0.40 | 13.90 ± 0.55 |
| 11.8-14.8 | | | |
| Hematocrit (%) 36.2- | 37.96 ± 1.19 | 39.43 ± 1.62 | 44.12 ± 1.85 |
| 47.2 | | | |
| MCV (fl) 73.6-82.1 | 76.50 ± 1.80 | 77.53 ± 2.79 | 79.64 ± 1.76 |
| MCH (pg) 23.3-26.0 | 24.76 ± 0.42 | 24.83 ± 1.08 | 25.08 ± 0.53 |
| MCHC (g/dl) 31.2- | 32.32 ± 0.57 | 32.05 ± 0.33 | 31.52 ± 0.38 |
| 33.1 | | | |
| RDW (%) 10.9-12.8 | 11.42 ± 0.45 | 12.03 ± 0.68 | 12.14 ± 0.49 |

TABLE 5
Clinical hematology reference values at 12 weeks of age for
LCPUFA supplemented term baboon neonates (range, mean ± SD.

| Diet | C | L | L3 |
|---|---|---|---|
| WBC (×10³) 1.2-7.9 | 4.44 ± 2.01 | 6.23 ± 1.54 | 5.24 ± 1.36 |
| RBC (×10⁶) 4.36-5.46 | 4.80 ± 0.23 | 4.95 ± 0.50 | 4.85 ± 0.24 |
| Hemoglobin (g/dl) | 11.74 ± 0.64 | 12.13 ± 0.76 | 12.28 ± 0.64 |
| 10.9-12.8 | | | |
| Hematocrit (%) 33.8- | 36.28 ± 1.16 | 37.43 ± 2.86 | 38.06 ± 1.80 |
| 40.0 | | | |
| MCV (fl) 72.1-81.4 | 75.68 ± 1.86 | 75.65 ± 2.44 | 78.40 ± 1.87 |
| MCH (pg) 23.5-26.0 | 24.46 ± 0.71 | 24.53 ± 0.89 | 25.30 ± 0.51 |
| MCHC (g/dl) 31-33.1 | 32.32 ± 0.83 | 32.40 ± 0.52 | 32.26 ± 0.30 |
| RDW (%) 11-12.7 | 11.70 ± 0.51 | 11.68 ± 0.30 | 12.10 ± 0.53 |

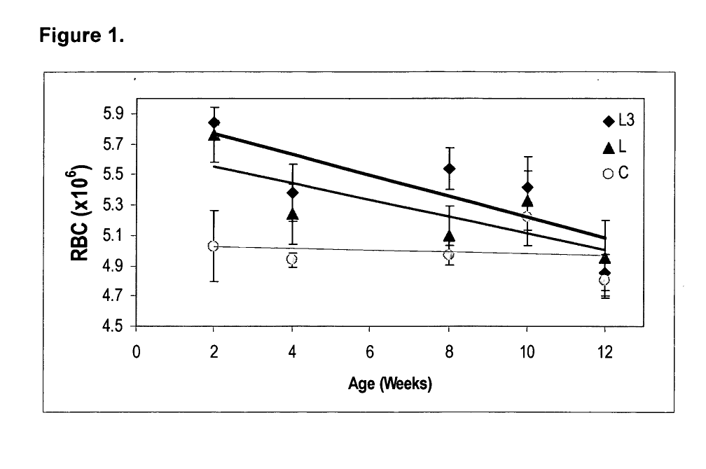

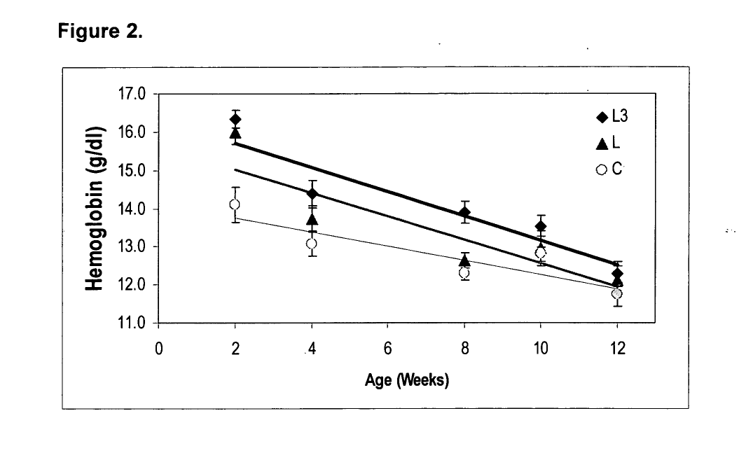

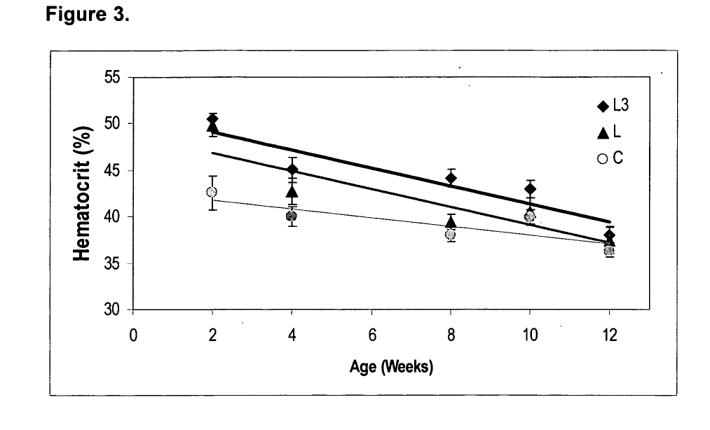

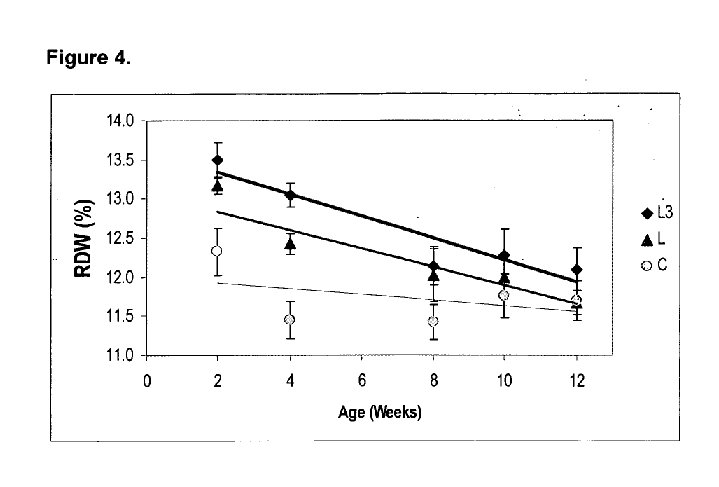

Significant differences due to supplementation were observed for several measurements (FIGS. 1-4). LCPUFA elevated values for RBC, hematocrit, hemoglobin, and RDW and the highest levels were seen in L3 group, followed by the L and C diet groups. RBC and hemoglobin values fell from 5.5±0.5×10⁶ and 15.34±1.26 g/dl to 4.9±0.3×10⁶ and 12.04±0.67 g/dl at 12 weeks, respectively. Initial blood measurements indicate significant effects of dietary LCPUFA fed from birth. Regression equations revealed consistent trends in intercepts, with higher initial values for L3 and L compared to the unsupplemented C group.

At 2 weeks of age, RBC, hemoglobin and hematocrit measurements were highest in the L3 group (5.8±0.03×10₆, 16.3±0.5 g/dl, 50.5±0.2%) while C was nearly 15% lower at 5.0±0.5×10₆, 14.1±0.9 g/dl, 42.6±3.7%, respectively. DHA and ARA supplementation also influenced the rate of decline in these blood parameters. Longitudinal changes in red cell measures were significantly different from the unsupplemented control group and L3 showed the most pronounced decrease over time, followed by the L group. All animals reached similar values at the 12 week nadir and significant differences were no longer observed for RBC, hemoglobin, hematocrit and RDW. Notable patterns in red cell indices MCV and MCH depict elevated values in the L3 diet group followed by L and C groups, a consistent but non-significant trend. Statistical differences between diet treatments were not observed for MCHC measurements.

Discussion of Results:

Age appropriate baboon hematology reference ranges are available for MCV, MCH, and MCHC and are similar to the present data. Havill, L. M., et al., Hematology and Blood Biochemistry in Infant Baboons (Papio Hamadryas), J. Med. Primatol 32:131-138 (2003). Declining red cell measurements during the first postnatal months are consistent with other published normal baboon values. Baboon hematological development follows trends documented in healthy human term infants. Postnatally, human infants reach a physiological nadir in RBC, hemoglobin and hematocrit at approximately 2 months. At 3 months, baboon hemoglobin concentrations decreased to 12.04±0.67 g/dl and would have eventually attained lowest values around 4 months of age. Besides species variability, blood count values change depending on collection site and differences may have been magnified due to sampling sites, human heel puncture versus baboon venipuncture.

Red cell indices during the first day of life change rapidly, and baboon cord or baseline blood information was not collected. Normally distributed measurements were assumed at parturition and experimental infant formulas were fed within 24 hours of birth. Initial blood samples were obtained at 2 weeks of age and significant differences in hematological indices were apparent between supplemented and unsupplemented neonates.

The effects of dietary LCPUFA on hematological parameters were evaluated by comparing results from L and L3 groups to the unsupplemented C group. DHA- and ARA-supplemented animals maintained significantly elevated RBC, Hb, and hematocrit values during the first weeks after birth and followed similar rates of decline compared to the C group. Regression slopes for these red cell parameters were remarkably consistent, steep L and L3 regression slopes contrasted by the more moderate slope of the unsupplemented group. Clear improvements of red cell indices were seen at higher concentrations of DHA. Although neonatal blood measurements eventually fell to similarly low values, the results show a potentially protective mechanism of baboons supplemented with LCPUFAs during the “physiologic anemia of infancy.” Elevated RBC and hemoglobin levels enhance oxygenation of body tissues, and while these effects were no longer significant at 12 weeks of age, they reveal surprising benefits of dietary DHA and ARA on postnatal erythropoiesis.

RDW is a calculation of the variation in red cell size and regression analysis detected significant differences in supplemented infants compared to the control group. While animals consuming dietary LCPUFAs had slightly greater variation in cell size, values were within normal ranges and the role of RDW values in diagnosis is still uncertain. Elevated hematocrit and RBCs suggest an actual increase in the number of red cells in whole blood and possibly increased production of new cells. Reticulocytes, RBC precursors, are larger in size than mature red cells. If RBCs were elevated due to increased production of cells, the newly released reticulocytes would have influenced RDW measures. However, blood smears were not analyzed and reticulocyte information was not available.

Dietary LCPUFAs are known to alter RBC and tissue fatty acid profiles in animal and human neonates. Lipid composition of erythrocyte membranes are ˜50% by weight, predominately in the form of phospholipids. A potential explanation for elevated red cell parameters of supplemented animals may be increased RBC survival. The normal life span of adult red cells is approximately 120 days and RBCs created during last months of fetal life range between 45-70 days. Erythrocytes from term infants survive around 60-80 days, while those of premature infants are considerably shorter. Alterations in membrane function are thought to be responsible for the decreased survival of fetal RBCs. Normal neonatal red cells tend to be less flexible and more resistant to lysis, but more susceptible to oxidant induced injury than adult cells. Incorporation of LCPUFA into blood cell membranes may have enhanced flexibility and vascular integrity to withstand stresses in circulation for enhanced survival.

Simultaneous changes in hemoglobin may contribute to observed improvements in red cell indices of supplemented neonates. During gestation, fetal hemoglobin begins switching to adult hemoglobin and continues 6 months postnatally. Related changes regulating hemoglobin-oxygen affinity and red blood cell 2,3-diphosphoglycerate (DPG) concentrations are initiated at birth. Fetal RBCs demonstrate a higher affinity for oxygen and lower affinity for 2,3-DPG, the protein that binds deoyxhemoglobin to facilitate oxygen release to body tissues. As infants mature, fetal hemoglobin declines, erythrocyte interaction with 2,3-DPG improves and a corresponding right shift in the hemoglobin-oxygen dissociation curve occurs.

The liver plays a critical role in carbohydrate and lipid metabolism and iron homeostasis. LCPUFA supplementation has been shown to increase liver DHA concentrations in neonatal baboons. Additional changes during the perinatal period may influence absorption or transport of nutrients and maturation of the hematopoietic system. Fetal blood production begins in the liver, gradually shifting to bone marrow during the last 3 months of gestation and continues 1 week postnatally.

Production of erythropoietin (EPO), an essential growth factor responsible for prolonging RBC cell survival and stimulating erythroid proliferation, also occurs in the fetal liver. EPO production transitions to the peritubular cells of the kidneys during the first months of life. In neonatal sheep, the transition is completed around 40 days after birth. The adult kidney produces EPO in response to hypoxia and is more sensitive to fluctuations in oxygen. At birth, the sudden increase in oxygen tension initiates several changes that include decreased hematopoiesis, reticulocyte count, marrow erythroid elements, and EPO suppression. EPO production declines for 4-6 weeks until adult concentrations are attained around 10-12 weeks of age. EPO is eliminated faster in neonates, with human infant plasma EPO levels lowest during the first postnatal month. Amniotic fluid and human breast milk both contain EPO. EPO receptors have been identified in the gastrointestinal tract, endothelial cells, spleen, liver, kidney, lung, spinal cord, and brain suggesting non-hematopoietic roles for EPO.

The liver stores excess iron and produces transferrin, a protein bound to all circulating plasma iron. Iron homeostasis is a complex and tightly regulated process, controlled at the level of absorption in the small intestine. No mechanism for iron excretion exists and accumulation is dangerous, due to oxygen free radical production. The recent discovery of the hormone, hepcidin, has implicated the liver in regulation of intestinal iron absorption. Hepcidin inhibits iron absorption and its production decreases during iron deficiency and increased erythropoiesis. Iron status is thought to play a role in the signaling expression of EPO and we propose an explanation for early hematological differences in LCPUFA supplemented animals based on fatty acid interactions with EPO and iron availability. Dietary DHA and ARA help to promote the liver to kidney EPO transition, moderately increasing levels of EPO. EPO receptors in the bone marrow, gastrointestinal tract and other parts of the body sense the circulating EPO, subsequently stimulating red cell production and maturation of the intestinal mucosa.

Iron absorption becomes more efficient and readily available for hematopoiesis, complemented by simultaneous changes in red cell membranes and the liver. Iron deficient red cell membranes are abnormally rigid and the unsupplemented C group may have required iron from products of red cell breakdown. While all baboon neonates consumed formula containing the same amount of iron, absorption would have depended greatly on gastrointestinal tract maturity. EPO may have interacted with other growth factors to promote maturation of crypt cells in the villi.

In the developing neonatal rat intestine, EPO increases small bowel length and villus surface area. Human studies have found less severe necrotizing enterocolitis (NEC) in infants fed formulas supplemented with DHA and ARA and a retrospective study examining very low birth weight infants reported lower incidence of NEC when recombinant EPO was administered. A randomized trial in preterm infants treated with recombinant EPO and iron had higher hematocrit and reticulocyte count and fewer blood transfusions compared to infants treated with EPO alone.

During the first weeks of postnatal growth, supplementation with ARA and increasing levels of DHA revealed unexpected but consistent patterns in hematological measurements. Improvements in red cell indices of supplemented animals provide physiological advantages and accelerated erythropoiesis during early development. These findings capture specific changes during a dynamic period that have not been reported in previous infant supplementation studies with more limited blood collection. Similar studies examining LCPUFA supplementation and cognitive function in human infants have also shown initial developmental advantages that dissipate at later ages. This pattern is thought to “reflect some developmental cascade in which an early developmental advantage in one cognitive domain gives rise to advantages in other, higher order domains.” Colombo, J., et al., Maternal DHA and the Development of Attention in Infancy and Toddlerhood, Child Dev. 57:1254-1267 (2004). Blood indices provide glimpses of rapidly developing processes in neonates and it is believed that accelerated erythropoiesis may have lasting effects extending beyond hematopoiesis.

The influence of dietary LCPUFA on ontogeny of hematological profiles in term baboon neonates was assessed. Hematological values were similar to established infant baboon reference ranges and consistent with increasing maturity documented during human neonatal development. During the first postnatal weeks, supplementation at levels of 0.32% DHA/0.64% ARA and 0.96% DHA/0.64% ARA increased RBC, hemoglobin and hematocrit values by 12% and 15%, respectively when compared to an unsupplemented control group. Infant formulas supplemented with LCPUFAs promote accelerated erythropoiesis and gastrointestinal maturation to prevent the rapid decline in red cell measurements associated with neonatal anemia.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for preventing or treating anemia in a subject, the method comprising administering to the subject a therapeutically effective amount of DHA and ARA.

2. The method according to claim 1, wherein the anemia is selected from the group consisting of hemolytic anemia, microangiopathic hemolytic anemia, hyperspienism, anemia caused by a pyruvate kinase deficiency, dyserythropoietic anemia, spherocytosis, sideroblastic anemia, autoimmune hemolytic anemia, sickle cell anemia, thalassemia, Glucose-6-phosphate dehydrogenase (G6PD)-deficient anemia, pernicious anemia, aplastic anemia, anemia caused by liver disease or renal disease, and anemia caused by various vitamin or nutrient-deficiencies, such as vitamin B12, B2, B6, C, A, D, E, or K, iron, folic acid, zinc, copper, calcium, or protein.

3. The method according to claim 1, wherein the subject is in need of such treatment.

4. The method according to claim 1, wherein the subject is in need of such prevention.

5. The method according to claim 1, wherein the therapeutically effective amount of DHA is between about 3 mg per kg of body weight per day and 150 mg per kg of body weight per day.

6. The method according to claim 1, wherein the therapeutically effective amount of DHA is between about 15 mg per kg of body weight per day and 60 mg per kg of body weight per day.

7. The method according to claim 1, wherein the therapeutically effective amount of ARA is between about 5 mg per kg of body weight per day and 150 mg per kg of body weight per day.

8. The method according to claim 1, wherein the therapeutically effective amount of ARA is between about 15 mg per kg of body weight per day and 90 mg per kg of body weight per day.

9. The method according to claim 1, wherein the therapeutically effective amount of ARA is between about 20 mg per kg of body weight per day and 60 mg per kg of body weight per day.

10. The method according to claim 1, wherein the ratio of ARA:DHA by weight is from about 1:3 to about 9:1.

11. The method according to claim 1, wherein the ratio of ARA:DHA by weight is about 2:1.

12. The method according to claim 1, wherein the ratio of ARA:DHA by weight is about 1:1.5.

13. The method according to claim 1, wherein the DHA and ARA are administered to an infant.

14. The method according to claim 13, wherein the DHA and ARA are administered to the infant during the time period from birth until the infant is about one year of age.

15. The method according to claim 13, wherein the DHA and ARA are administered to the infant in an infant formula.

16. The method according to claim 15, wherein the infant formula comprises DHA in an amount of from about 2 mg to about 100 mg per 100 kcal infant formula.

17. The method according to claim 15, wherein the infant formula comprises DHA in an amount of from about 5 mg to about 75 mg per 100 kcal infant formula.

18. The method according to claim 15, wherein the infant formula comprises DHA in an amount of from about 15 mg to about 60 mg per 100 kcal infant formula.

19. The method according to claim 15, wherein the infant formula comprises ARA in an amount of from about 4 mg to about 100 mg per 100 kcal infant formula.

20. The method according to claim 15, wherein the infant formula comprises ARA in an amount of from about 10 mg to about 67 mg per 100 kcal infant formula.

21. The method according to claim 15, wherein the infant formula comprises ARA in an amount of from about 30 mg to about 40 mg per 100 kcal infant formula.

22. The method according to claim 15, wherein the infant formula contains substantially no EPA.

23. A method for preventing or treating anemia in a subject, the method comprising administering to the subject a therapeutically effective amount of DHA.

24. A method for increasing the red blood cell count in a subject, the method comprising administering to the subject a therapeutically effective amount of DHA and ARA.

25. A method for increasing the hemoglobin concentration in a subject, the method comprising administering to the subject a therapeutically effective amount of DHA and ARA.

26. A method for elevating hematocrit values in a subject, the method comprising administering to the subject a therapeutically effective amount of DHA and ARA.

27. A method for promoting accelerated erythropoiesis in an infant, the method comprising administering to the infant a therapeutically effective amount of DHA and ARA.

28. A method for enhancing the ability of a subject to absorb iron, the method comprising administering to the infant a therapeutically effective amount of DHA and ARA.

29. The use of DHA and ARA for the preparation of a medicament for the prevention or treatment of anemia.
